(19) United States
(12) Patent Application Publication
(10) Pub. No.: US 2020/0147029 A1
(43) Pub. Date: May 14, 2020

(54) NANOSTRUCTURED AND BIOCOMPATIBLE BIOCATALYSTS FOR USE IN CANCER TREATMENT

(71) Applicant: Wenceslao GÓMEZ-LÓPEZ, Mexico City (MX)

(72) Inventor: Tessy María LÓPEZ-GOERNE, Mexico City (MX)

(21) Appl. No.: 16/632,846

(22) PCT Filed: Jul. 18, 2018
(86) PCT No.: PCT/IB2018/055335

§ 371 Date: Jan. 21, 2020

Related U.S. Application Data
(60) Provisional application No. 62/534,748, filed on Jul. 20, 2017.

(51) Int. Cl. A61K 31/282; A61K 33/243; A61K 9/00; A61K 9/14; A61K 9/51

(52) U.S. Cl. CPC A61K 31/282; A61K 33/243; B82Y 5/00; A61K 9/143; A61K 9/5115; A61K 9/0024

(57) ABSTRACT

Nanostructured and biocompatible biocatalysts an organic ligand either linear or branched to Pt, Cu, or Fe-based compounds, in II, III or IV oxidation state, having cytotoxic activity for use in the treatment of cancer in animals or humans.

### FIELD OF THE INVENTION

This invention relates to the use of nanostructured and biocompatible biocatalysts in the treatment of cancer.

### BACKGROUND

Cancer is one of the leading causes of death all over the world. The treatment use is surgery, radiotherapy, chemotherapy or a combination of them. Chemotherapy uses chemical agents (anticancer drugs) to kill cancer cells, is one of the primary methods to cancer treatment. Unfortunately, most of the anticancer drugs have limited selectivity for cancer and are inherently toxic to both cancer and normal tissues. Like other cancer chemotherapeutic agents, compounds that exhibit high antitumor activity such as cis-platin are typically highly toxic. The main disadvantages of cis-platin are its extreme nephrotoxicity and neurotoxicity, which is an important limiting factor to use. Its rapid distributed via blood stream, with a circulation half life of only a few minutes, and its strong affinity to plasma proteins (Freise et al. 1982 Arch. Int. Pharmacodyn Ther. 258(2): 180-192). Other side effects of anticancer drugs include the decrease of white blood cells, red blood cells and platelets increasing the risk of infections, bruising and bleeding.

Most important, conventional treatments may cause drug resistance and hence treatment failure (Pastan and Gottesman, 1991, Gottesman 2002). A major mechanism of resistance is related to the P-glycoprotein pump located in cell membrane (Gottesman 2002) that binds drugs as they enter the plasma membrane transporting the drug out of the cells. As a consequence the effective drug concentration in the cytoplasm is well below the cell-killing threshold, resulting in a limited therapeutic efficacy.

The development of new methodologies that have higher drug selectivity for cancer and simultaneously reduce toxicity to healthy tissues is a major challenge in cancer treatment. The cancer cutoff size of cancer's blood capillaries (ca. 400-800 nm) allows extravasations of colloid particles to cancer tissues on the other hand as cancer tissues have fewer lymphatic capillaries drainage from these capillaries to healthy tissues is reduced causing the trapping of colloidal particles in cancer tissues, this is referred as the “enhanced permeability and retention effect” (Maeda et al. 2001, Lukyanov et al. 2002). Nanoparticles fabricated by self-assembling of amphiphilic copolymers have been used as carriers for cis-platin (Yokoyama et al. 1996, Bogdanov et al. 1997).

The use of inorganic oxides nanoparticles offers a suitable mean to deliver drugs to tissues or cells. Their submicrometric size favors the taken up by cells via endocytosis/phagocytosis, the hydrophilic character of their surfaces allows evading the recognition by the reticuloendothelial systems and their intrinsic stability prevents the breakage in the bloodstream. In addition to this, they may have high surface area a controlled pore size distribution and if required tailored surface acid-base properties for adapting them to site specificity.

State of the art research in the treatment of chronic diseases is based on the development of controlled release systems capable of delivering drugs rapidly and efficiently to where they are needed. A major requirement is that these devices should insure delivery and penetration of the drug to the active site. New nanostructured materials represent an efficient way to administer medications and biological products in future applications. Hydrogels based on n-isopropylacrilimide and methacrylic acids (MAA) have recently received considerable attention. This is due to their ability to swell in response to the stimulation of the medium. In the solid state, the existence of interpolymeric complexes in which monomers are linked together through hydrogen bonds has been observed. These linkages occur under acid conditions and are stabilized through hydrophobic interactions. This leads to a marked dependence on the pH of the medium in which swelling occurs. This swelling is also strongly dependent on the degree of cross-linking. The use of drug delivery by oral means has received considerable attention, particularly in cases in which activation is controlled by variations in the pH. Copolymers having a high concentration of N-isopropylacrilamide appear to be the most effective in enabling one to obtain different cut-off curves used in the drug model.

In the majority of cases, which involve controlled drug release, the medication or other biological agent, is introduced into the interior of the reservoir normally known as the transporter. The transporter usually consists of a polymeric material. Under normal conditions the rate of drug release is controlled by the properties of the polymeric material which constitutes the transporter. However, other factors may also be rate determining. When these factors are taken into account, it may be possible to insure a slow, constant rate of drug delivery over extended periods of time. The use of these materials has lead to considerable advances in drug delivery when compared to systems currently in use. In conventional drug delivery systems, drug concentrations reach a maximum value only to decay, finally reaching a concentration, which requires the administration of another dose. Additionally, if the maximum drug concentration exceeds the safe level or if, alternatively it falls below the required dose, cyclic periods will occur during which the drug is not producing the desired effect. This is generally known as “variations in tisular exposure”. When controlled drug release is used, it may be possible to maintain drug concentrations, which fall between the maximum allowed rate, and the minimum concentration at which the rate is effective.

When dealing with inorganic oxide nanoparticles, the sol-gel technique with or without the use of templates can be used as a good method by which the various solid phases can be controlled (T. Lopez et.al., Catalysis Today 35, 293.1997). A greater degree of control can be achieved in comparison to other methods of synthesis. One can tailor make the reservoir to fit specific applications by using this method. Advances include: Superior homogeneity and purity; High solid acidity; High biocompatibility with any tissue; Better nano and microstructural control of the inorganic oxide matrices; Greater BET surface area; High dispersion of the platinum on the matrices; Improved thermal stability of the drugs attached to the transporter; Well-defined mean pore size distributions; Inorganic chain structures can be generated in solution; A finer degree of control over the hydroxylation of the transporter can be achieved.

The process of transporter fabrication has as an aim the optimization of the following variables: particle size, mean pore size, interaction forces and the degree of functionalization. It may also be desirable to modify the textural and electronic behavior of the transporter.

Sol-gel technology is an important synthesis method by which the crystalline phases and particle size of inorganic hydrous oxides can be controlled. A sol is a fluid, colloidal dispersion of solid particles in a liquid phase where the particles are sufficiently small to stay suspended in Brownian motion. A “gel” is a solid consisting of at least two phases wherein a solid phase forms a network that entraps and immobilizes a liquid phase. In the sol-gel process the dissolved or “solution” precursors can include metal alkoxides, alcohol, water, acid or basic promoters and on occasion salt solutions. Metal alkoxides are commonly employed as high purity solution precursors. When they react with water through a series of hydrolysis and condensation reactions they yield amorphous metal oxides or oxo-hydroxide gels. When the volatile alcohol molecules are removed the result is the formation of crystalline solid compounds. This solid can be modified by adding suitable amounts of desired molecules during the synthesis process whose amount an stability are determined by the stability constant.

The materials that are used as colloid precursors can be metals, metal oxides, metal oxo-hydroxides or other insoluble compounds. The degree of aggregation or flocculation in the colloidal precursor can be adjusted in such a way that the pore size distribution can be controlled. Dehydration, gelation, chemical cross-linking and freezing can be used to form the shape and appearance of the final product. Some advantages using sol-gel technology include control over the purity of the alkoxide precursors, control over the homogeneity of the product, control over the evolution of the desired crystalline phases and most importantly, the reproducibility of the materials synthesized.

The hydrolysis product is not fully hydrolyzed nor can it ever be a pure oxide. It can be in the form, MₙO_{2n-(x+y)/2}(OH)ₓOR)_{y}, M stands for silicon, titanium or a mixture of both and R for an organic fragment, preferably CₙHₙ₊₁, either linear or branched, where n is the number of titanium atoms polymerized in the polymer molecule and x and y is the number of terminal OH and OR groups respectively. It is well known that some sol-gel structures attain their highest coordination state through intermolecular links (Sankar G., Vasureman S, and Rao C.N.R., J. Phys. Chem, 94, 1879 (1988) y otras más modernas). Because there are strong chemical interaction forces between the drugs and the inorganic nanoparticle transporter, it is possible to encapsulate a large amount of medication within the transporter.

Additional titania patents using sol-methods:

U.S. Pat. No. 6,124,367. This patent protects reservoirs used in the Fischer Tropsch reactions from sintering by imparting a higher degree of mechanical strength to the reservoir. It incorporates SiO₂ and Al₂O₃ into the reservoir and claims a rutile-anatase ratio of 1/9. It is a porous reservoir with either a spherical or a cylindrical shape. It is made by extrusion, spray drying or tableting.

U.S. Pat. No. 6,117,814. This patent describes a titania reservoir which also incorporates silica and alumina as a binder into the structure. The purpose of the binder is to impart better mechanical properties to the reservoir. The size range of this reservoir is from between 20 to 120 microns. The reservoir is approximately 50% binder, which is fabricated by a sol-gel process.

U.S. Pat. No. 6,087,405. This patent describes a reservoir to be used in a Fischer Tropsch gas synthesis reaction. The reservoir incorporates group VII metals into its structure. The rutile-anatase ratio in the structure is a distinguishing feature of this patent.

### OBJECTIVES

1. The development of nanostructured materials for use as biocatalyst in treatment of cancer.

2. Obtain and optimize the nanostructure biocatalyst capable to kill maligned cells by means catalytic reaction.

3. Optimization of materials to enable control of the following parameters: pore size distribution, contact area, structure, electronic density, particle size, crystalline phase, degree of functionalization, diffusion, size of biocatalyst required to react with the cell. the drug, and release time for effective delivery.

4. Obtain an effective nanoparticle to use in cancer therapy and to prevent a side effects on the blood stream, liver, intestine and kidneys.

### SUMMARY OF THE INVENTION

Nanostructured and biocompatible biocatalysts an organic ligand either linear or branched to Pt, Cu, or Fe-based compounds, in II, III or IV oxidation state, having cytotoxic activity for use in the treatment of cancer in animals or humans.

### DESCRIPTION OF THE DRAWINGS

FIG. 1 is (a) X-ray diffraction pattern and, (b) FTIR spectrum of Pt/SiO₂—Pt(NH₃)₄Cl₂.

FIG. 2 is a transmission electron microscopy of the nanostructured particles, which comprise the Pt/SiO₂—Pt(NH₃)₄Cl₂ biocatalyst.

FIG. 3 are photomicrographs of hematoxylin and eosin stained sections of (a) tumor treated with Pt/SiO₂—Pt (NH₃)₄Cl₂ nanoparticles, (b) higher amplification, and, (c) TUNNEL analysis.

### DETAILED DESCRIPTION

This invention is related to the synthesis of nanostructured inorganic nanostructured and biocompatible biocatalysts defined as MnO_{2n-(x+y)/2}(OH)ᵥ(SO₄)_{w}(PO₄)ₓ(OR)_{y}(Cl)_{z} where M stands for silicon, titanium or a mixture of both and R for an organic ligand, preferably CₙHₙ₊₁, either linear or branched to Pt, Cu, or Fe-based compounds, in II, III or IV oxidation state, having cytotoxic activity. These nanostructured biocatalysts are directly administered into the tumor. The matrix acidity, structure, electronic density, pore size distribution, matrix particle size, platinum, copper or iron particle size, platinum, copper or iron dispersion on the support (silica or titania), crystallite size and oxidation state of platinum, copper or iron are controlled. These anticancer biocatalyst formulations will be delivered directly into the tumor.

The present invention includes a novel nano-material (silica, titania and silica-titania) obtained by the sol-gel process to which platinum compounds are bound. The support particle size ranges between 10 nm to 1 μm.

The platinum metal is either bound as metallic nanoparticles or covalently bound platinum complexes. The metal nanoparticle size ranges from atomic dispersion to 100 nm.

This nanomaterial consists of partially hydrolyzed oxides having a Ti:Si range of compositions between (100:0 and 0:100). These materials were prepared using a sol-gel process, which has been used to synthesize ceramic and glass materials.

The titania, silica and titania-silica xerogels (100:0, 0:100) materials are found to be biocompatible with surrounding tissue.

The synthesis of the platinum containing drug is carried out by adding the platinum compound during the gelation process or by grafting the platinum compound to the sol-gel obtained oxides. The total amount of platinum can be as high as 10% by weight.

Mesoporous sol-gel oxides can be synthesized, in reactive (i.e. air, carbon dioxide, etc.) or inert atmosphere (i.e nitrogen, argon, etc.) at pH ranging from 2 to 12 using water:alkoxide ratio ranging from 2 to 64. Water, C₁ to C₅ primary, secondary or tertiary alcohols, acetyl acetone, acetone or a mixture alcohol-water or acetone-acetyl acetone was used as solvent for the synthesis.

The pH during the synthesis was fixed using HCl, H₂SO₄, H₃PO₄ carboxylic acids (i.e. EDTA, acetic acid, -amino butyric acid, glutamic acid, etc) or bases (i.e. ammonium hydroxide, phenitoine, puric bases, pyrimidic bases, etc)

The gelation process was carried out from room temperature to 80° C. in the presence or absence of organic templates or modifiers (i.e. P123, acetylacetone, CTAB, etc).

Platinum compound precursors are H₂PtCl₆ cis-Pt or PtAcAc or Pt(NH₃)₄Cl₂.

Pore volumes and pore diameters are not strongly affected by platinum compound loadings.

The administration form can be: a) nanoparticle suspension in physiological compatible fluids; b) extrudates, in this case biocompatible binders might be used (i.e. poly[bis(p-carboxypenoxy)]propane-sebacic acid, PLGA, methylcellulose, PVP, etc); and c) implantable self-supported nanodevices.

The present disclosure includes disclosure of a formulation, comprising a quantity of a silica oxide, a quantity of a titanium oxide, and a quantity (or quantities) of one or more of copper, silver, gold, iron, rutenium, palladium, zinc, manganese, iridium and/or platinum metals, as referenced herein.

The sol-gel methodology is used to control the physico-chemical properties of the material in a thin, nanometric size and with a wide surface area. The nanoparticle comprised in the disclosed formulation is characterized by being a solid acid consisting of mixed oxides of silica and titania incorporating in its dispersed matrix, copper, silver, gold, iron, rutenium, palladium, zinc, manganese, iridium and/or platinum metals, or mixtures thereof, to minimum concentrations; and at least one functionalizing agent in contact with the particle. The carrier may be in liquid, oil, gel or solid form.

Sol-gel technology is an important synthesis method by which the crystalline phases and particle size of inorganic hydrous oxides can be controlled. A sol is a fluid, colloidal dispersion of solid particles in a liquid phase where the particles are sufficiently small to stay suspended in Brownian motion. A “gel” is a solid consisting of at least two phases wherein a solid phase forms a network that entraps and immobilizes a liquid phase. In the sol-gel process the dissolved or “solution” precursors can include metal alkoxides, alcohol, water, acid or basic promoters and on occasion salt solutions. Metal alkoxides are commonly employed as high purity solution precursors. When they react with water through a series of hydrolysis and condensation reactions they yield amorphous metal oxides or oxo-hydroxide gels. When the volatile alcohol molecules are removed the result is the formation of crystalline solid compounds. This solid can be modified by adding suitable amounts of desired molecules during the synthesis process, wherein amount and stability are determined by the stability constant.

The materials that are used as colloid precursors can be metals, metal oxides, metal oxo-hydroxides or other insoluble compounds. The degree of aggregation or flocculation in the colloidal precursor can be adjusted in such a way that the pore size distribution can be controlled. Dehydration, gelation, chemical cross-linking and freezing can be used to form the shape and appearance of the final product. Some advantages using sol-gel technology include control over the purity of the alkoxide precursors, control over the homogeneity of the product, control over the evolution of the desired crystalline phases and, most importantly, the reproducibility of the materials synthesized.

The hydrolysis product is not fully hydrolyzed nor can it ever be a pure oxide. It can be in the form, MnO_{2n-(x+y)/2}(OH)ₓOR)_{y}, wherein M stands for silicon, titanium or a mixture of both and R for an organic fragment, preferably CₙHₙ₊₁, either linear or branched, wherein n is the number of titanium atoms polymerized in the polymer molecule and x and y is the number of terminal OH and OR groups respectively. It is well known that some sol-gel structures attain their highest coordination state through intermolecular links. Because there are strong chemical interaction forces between the drugs and the inorganic nanoparticle transporter, it is possible to encapsulate a large amount of medicament within the transporter.

### Sol-Gel Process Using Metal Alkoxides:

At the functional group level, three reactions are generally used to describe the sol-gel process: hydrolysis, alcohol condensation, and water condensation. However, the characteristics and properties of a particular sol-gel inorganic network are related to a number of factors that affect the rate of hydrolysis and condensation reactions, such as, pH, temperature and time of reaction, reagent concentrations, catalyst nature and concentration, H₂O/M molar ratio (R), aging temperature and time, and drying. Of the factors listed above, pH, nature and concentration of catalyst, H₂O/M molar ratio (R), and temperature have been identified as most important. Thus, by controlling these factors, it is possible to vary the structure and properties of the sol-gel-derived inorganic network over wide ranges. For example, Sakka et al. observed that the hydrolysis of TEOS utilizing R values of 1-2 and 0.01 M HCl as a catalyst yields a viscous, spinnable solution. It was further shown, that these solutions exhibited a strong concentration dependence on the intrinsic viscosity and a power law dependence of the reduced viscosity on the number average molecular weight⁽³¹⁻³⁴⁾:

[n]=k(Mn)a (1)

Values for a ranged from 0.5 to 1.0, which indicates a linear or lightly branched molecule or chain.

Values of “a” in eq. 1 ranged from 0.1 to 0.5, indicating spherical or disk shaped particles. These results are consistent with the structures which emerge under the conditions employed by the Ströber process, for preparing SiO₂ powders. It was further shown that with hydrolysis under basic conditions and R values ranging from seven (7) to twenty-five (25), monodisperse, spherical particles could be produced.

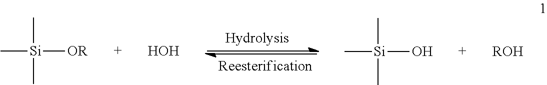

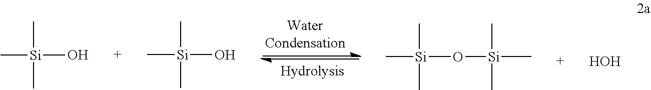

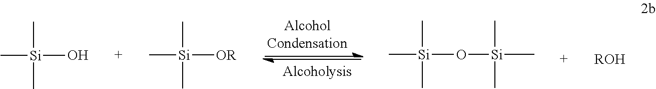

Generally speaking, the hydrolysis reaction (Eq. 2), through the addition of water, replaces alkoxide groups (OR) with hydroxyl groups (OH). Subsequent condensation reactions are made, involving the silanol groups (Si—OH) produce siloxane bonds (Si—O—Si) plus the by-products water or alcohol in the case of silica. Under most conditions, condensation commences before hydrolysis is complete. However, conditions such as, pH, H₂O/Si molar ratio (R), and catalyst can force completion of hydrolysis before condensation begins. Additionally, zo because water and alkoxides are immiscible, a mutual solvent is utilized. With the presence of this homogenizing agent, alcohol, hydrolysis is facilitated due to the miscibility of the alkoxide and water. As the number of siloxane bonds increases, the individual molecules are bridged and jointly aggregate in the sol. When the sol particles are aggregate, or inter-knit into a network, a gel is formed. Upon drying, trapped volatiles (water, alcohol, etc.) are driven off and the network shrinks as further condensation can occur. It should be emphasized, however, that the addition of solvents and certain reaction conditions may promote esterification and depolymerization reactions. The hydrolysis/condensation reaction follows two different mechanisms, which depend of the coordination of metallic central atom. When the coordination number is satisfied the hydrolysis reaction occurs by nucleophilic substitution (Sₙ):

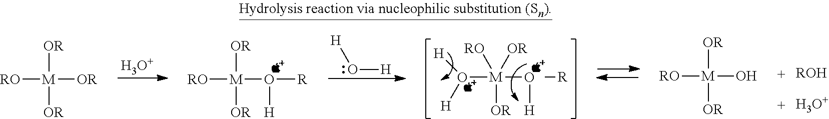

When the coordination number is major, the hydrolysis reaction takes place by nucleophilic addition:

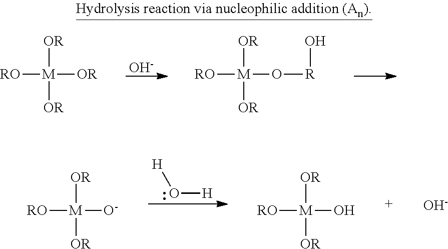

These mechanisms need that the oxygen coordination is increased from 2 to 3, the additional bond generation involves one electron pair of the oxygen and the new bond can be equivalent to the other bonds. During the condensation step an enormous concentration of hydroxyl groups are formed. This OH can be linked between the metallic atoms or only be simple —OH ligand in the surface.

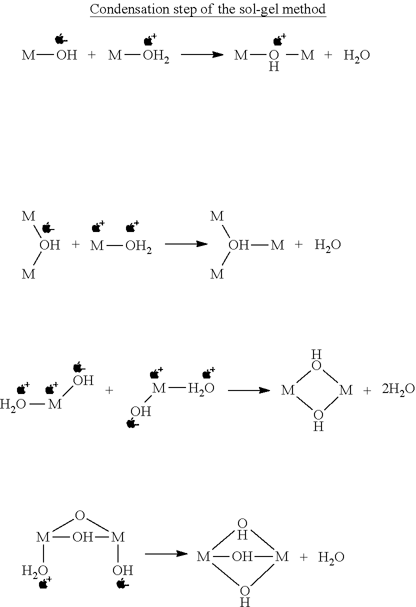

### Acid-Catalyzed Mechanism

Under acidic conditions, it is likely that an alkoxide group is protonated in a rapid first step. Electron density is withdrawn from the silicon atom, making it more electrophilic and thus more susceptible to attack from water. This results in the formation of a penta-coordinate transition state with significant SN₂-type character. 13 The transition state decays by displacement of an alcohol and inversion of the silicon tetrahedron, using silica as example:

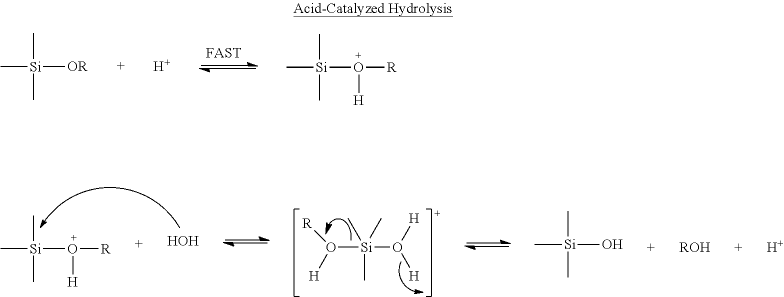

### Base-Catalyzed Mechanism:

Base-catalyzed hydrolysis of silicon alkoxides proceeds much more slowly than acid-catalyzed hydrolysis at an equivalent catalyst concentration. Basic alkoxide oxygens tend to repel the nucleophile, —OH. However, once an initial hydrolysis has occurred, following reactions proceed stepwise, with each subsequent alkoxide group more easily removed from the monomer then the previous one. Therefore, more highly hydrolyzed silicones are more prone to attack. Additionally, hydrolysis of the forming polymer is more sterically hindered than the hydrolysis of a monomer. Although hydrolysis in alkaline environments is slow, it still tends to be complete and irreversible. Thus, under basic conditions, it is likely that water dissociates to produce hydroxyl anions in a rapid first step. The hydroxyl anion then attacks the silicon atom. Again, an SN₂-type zo mechanism has been proposed in which the —OH displaces —OR with inversion of the silicon tetrahedron.

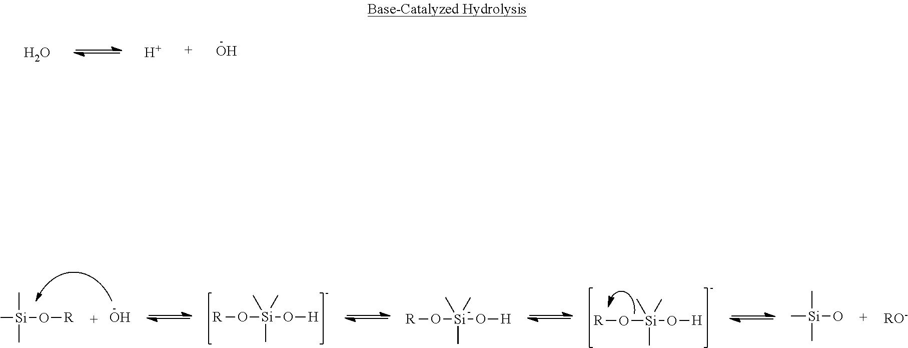

### Detailed Description of the Synthesis Methods Used:

Biocatalysts platinum, copper or iron compound-sol-gel synthesis: In the three-necked flask, a mixture consisting of deionized water, platinum, copper or iron compound, base or acid and solvent are refluxed. Prior to initiating the reflux, the pH of the solution is adjusted. In either case, the acid or the base is added in a “drop by drop” manner until the desired pH is obtained. The pH is monitored continually using a potentiometer throughout the entire process. Using a funnel, metal alkoxide or a mixture of metal alkoxides is added to the solution being refluxed. The dropwise addition is performed over a 4-10 hour period in order to enhance nucleation and functionalization. Following the addition of the alkoxide, the colloidal suspension is refluxed over a period from 24 to 240 hours. Following this process, the samples are dried under vacuum conditions in a roto-vapor (10⁻³ mm of Hg) in order to remove excess water and alcohol. Finally the samples are dried at 30° C. for 24-72 hours. In order to reach the final drying temperature of 30° C., the temperature is increased at a rate of 0.25° C./min to 5C./min using a conventional furnace.

In the case of mesostructured oxides, the synthesis procedure follows the known synthesis procedures for obtaining the adequate micelle concentration.

Alternatively, the inorganic oxides are synthesized following the same procedure but in the absence of the platinum, copper or iron compound. Once the nanomaterial is obtained the desired amount of platinum, copper or iron is added by:
a) A solution containing the platinum, copper or iron compound is added to the inorganic alkoxide in such a way that the solution volume matches the pore volume of the inorganic oxide.
b) A solution containing the platinum, copper or iron compound is added to the inorganic alkoxide at pH above or below the isoelectric point of the surface. In every case, the pH is adjusted to either preserve or decompose the platinum, copper or iron compound. For example for grafting [Pt (NH₃)₄]Cl₂ to a titania surface, a chloride rich solution at low pH is used.

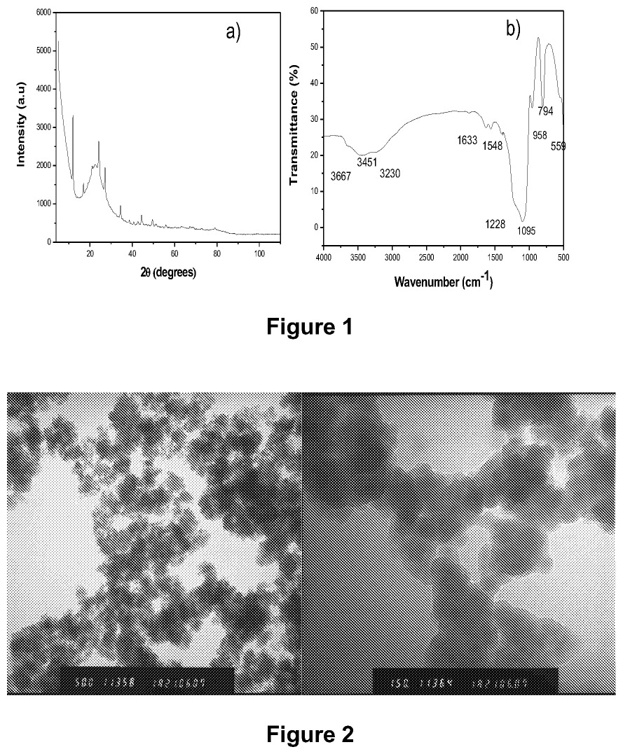

In FIG. 1a, an x-ray diffraction pattern, (obtained using a Brucker D-5000 instrument equipped with Cu-Ka radiation with a wavelength of 1.5418 A (45kV and 40mA)), in which an undefined broad band characteristic of amorphous silica is shown. Several small bands, which are reflections from the Pt (NH₃)₄Cl, centered at 12° and 24° (2 theta) are also observed. These results suggest that an OH group on the silica has been coordinated to Pt resulting in a square planar structure.

In the infrared transmittance spectrum showed in FIG. 1b, (the infrared spectra of the powdered samples was performed at room temperature using a Termo-Nicolet Nexus FT-IR spectrophotometer), a band centered at 3667cm⁻¹ is observed. This band is assigned to an OH stretching vibration which is interacting with the Pt complex. In general this band is observed at 3700 cm⁻¹ on pure silica and it is due to the presence of terminal hydroxyl groups which give rise to both Lewis and Brönsted acid sites. The band centered at 3451 cm⁻¹ is due to OH stretching vibrations, which are incorporated into the framework of silica. The corresponding OH bending vibrations are centered at 1633 cm⁻¹. The infrared bands associated with the stretching vibrations of the amine groups are observed at 3230 cm⁻¹. These observations are consistent with the fact that the complex has lost only one chlorine atom and that some decomposition of the complex has most likely occurred resulting in some PtO and supported metallic Pt. In the low energy region of the spectrum, a broad band centered at 1095 cm⁻¹ with a shoulder at 1228 cm⁻¹ is observed. These vibrations are due to stretching (—O—Si—O—) vibrations. The platinum precursor used in the synthesis, resulted in several new features observed in the infrared spectrum. In particular an H—N—H deformation band centered at 1548 cm⁻¹ and an asymmetric stretching band at 3230cm⁻¹ are evident.

In the micrographs shown in FIG. 2 (Zeiss, model MM 910 transmission electron microscope operating at 100 kv), the homogeneous morphology of the small agglomerates of spherical particles, around 30 nm in diameter, can be observed. On the left side of the figure the particle dimensions are clear. However, the right hand side gives a better idea of their distribution. Because Pt is very highly dispersed on the surface and has been cogelled with the tetraethoxysilane, it is not seen in the micrograph. Future studies using high resolution TEM will be focused on the identification of the Pt atoms on the support.

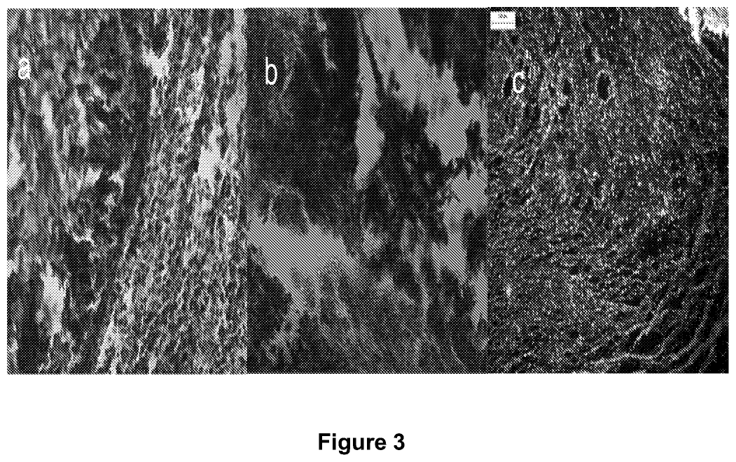

Histological studies using hematoxiline-eosine were performed on the tissue surrounding the trajectory of the injection of the suspension of Pt/SiO2-H₂PtCl₆ nanoparticles FIG. 3. The micrographs pertaining to this study tunnel are shown in FIG. 3. In FIG. 3a, an interface clearly shows a line of demarcation between two zones, one in which the tumor cells are clearly visible and the other, visibly showing the cell damage. In FIG. 3b, a higher magnification is used to examine the damaged area. In FIG. 3c the absence of growth in the tumoral tissue is apparent. The white dots are DNA fragments.

### EXAMPLES

### Example 1

To obtain 1 w/w % of platinum metal on TiO₂, 320 mg of Pt(NH₃)₄Cl₂.xH₂O was incorporated to a mixture containing 190 mL of ethanol and 29 mL of deionized water, under constant stirring at 343K. This mixture was refluxed for 10 minutes at 343K prior to the addition of the titanium alcoxide. Then 69 mL of the TiO₂ precursor, titanium n-butoxide, was added dropwise over a 4 h period. The resulting sols were maintained under constant stirring until gelation occurs. The total molar ratio water:alkoxide:alcohol was 8:1:16. Alter and aging period of 72 hours at room temperature xerogel samples were obtained by oven drying the obtained solids at 343K.

Table 1 shows the final volume of the tumours as a function of treatment. From this data it is clear that both the platinum coordination compound and the TiO₂ carrier produce a significant reduction of the tumour volume. This effect is greatly enhanced in the case of the groups treated with the TiO₂ and TiO₂—Pt nanodevices. In this later case, the tumour volume is just 44% of the volume achieved by the control group.

TABLE 1
Average tumour volume for the four designed groups of Wistar rats.

| | Volume/ |
|---|---|
| Treatment | cm³ |
| Control | 20.9 ± 4.9 |
| TiO₂—Pt(NH₃)₄Cl₂ | 47.2 ± 7.2 |
| TiO₂—cisPt | 26.7 ± 4.9 |
| SiO₂—Pt(NH₃)₄Cl₂ | 35.6 ± 8.5 |

1. Nanostructured and biocompatible biocatalysts defined as MnO_{n-(x+y)/2}(OH)ᵥ(SO₄)_{w}(PO₄)ₓ(OR)_{y}(Cl)_{z}, wherein M stands for silicon, titanium or a mixture of both and R for an organic ligand, preferably CₙHₙ₊₁, either linear or branched to Pt, Cu, or Fe-based compounds, in II, III or IV oxidation state, having cytotoxic activity for use in the treatment of cancer in animals or humans.

2. The nanostructured and biocompatible biocatalysts for use in the treatment of cancer of claim 1, wherein the particle size ranges between 10 nm to 1 μm.

3. The nanostructured and biocompatible biocatalysts for use in the treatment of cancer of claim 1, wherein the organic ligand is branched to Pt.

4. The nanostructured and biocompatible biocatalysts for use in the treatment of cancer of claim 3, wherein the platinum metal is either bound as metallic nanoparticles or covalently bound platinum complexes. The metal nanoparticle size ranges from atomic dispersion to 100 nm.

5. The nanostructured and biocompatible biocatalysts for use in the treatment of cancer of claim 3, wherein the platinum compound precursors are H₂PtCl₆ cis-Pt or PtAcAc or Pt(NH₃)₄Cl₂.

6. The nanostructured and biocompatible biocatalysts for use in the treatment of cancer of claim 1, wherein the mode of administration is: a) nanoparticle suspension in physiological compatible fluids; b) extrudates and c) implantable self-supported nanodevices.

7. The nanostructured and biocompatible biocatalysts for use in the treatment of cancer of claim 6, wherein the mode of administration is nanoparticle suspension in physiological compatible fluids.

8. The nanostructured and biocompatible biocatalysts for use in the treatment of cancer of claim 6, wherein the mode of administration is extrudates.

9. The nanostructured and biocompatible biocatalysts for use in the treatment of cancer of claim 6, wherein the mode of administration is implantable self-supported nanodevices.
